(19) United States
(12) Patent Application Publication
(10) Pub. No.: US 2003/0158507 A1
(43) Pub. Date: Aug. 21, 2003

(54) Preservation of blood platelets at cold temperatures

(76) Inventors: Vladimir Serebrennikov, Krasnoyarsk (RU); David Lucas, Lafayetta, CA (US); Luis H. Toledo, Portage, MI (US)

Correspondence Address: WILSON SONSINI GOODRICH & ROSATI, 650 PAGE MILL ROAD, PALO ALTO, CA, 943041050

(21) Appl. No.: 10/186,158

(22) Filed: Jun. 26, 2002

Related U.S. Application Data
(60) Provisional application No. 60/301,320, filed on Jun. 26, 2001.

(51) Int. Cl. A61K 47/00

(52) U.S. Cl. 604/4.01; 435/2

(57) ABSTRACT

Methods of cooling blood platelet suspensions which can be stored and preserved for extended periods of time. The normal morphology of platelets and their ability to function are substantially maintained. The steps include preparing a platelet suspension having blood platelets, a carbohydrate and at least one biocompatible polymer to assist in stabilizing platelet membranes. The platelet suspension may be cooled to a temperature of less than approximately 10 degrees C. at a rate of cooling greater than 1 degree C./min. The platelet suspension may be kept at a storage temperature ranging from approximately −1 to 6 degrees C. Additionally, methods are provided for maintaining the biological activity of blood platelets. Platelet suspensions may be initially prepared which include platelets, sucrose, verapamil, magnesium chloride and a biocompatible polymer. Cooling of the platelet suspension may be followed at a cooling rate ranging from approximately 1 to 12 degrees C./min or faster to a temperature below 10 degrees C. The cooled platelet suspension may be thus stored at a storage temperatures as high as 6 degrees C.

This patent application claims the benefit of the U.S. Provisional Patent Application Serial No. 60/301,320 filed on Jun. 26, 2001, which is incorporated by reference in its entirety herein.

### FIELD OF THE INVENTION

The present invention relates to methods for preserving blood platelets at refrigerator temperatures. More particularly, the invention relates to methods of cooling blood platelet suspensions which can be stored for extended periods of time while substantially maintaining the normal morphology of platelets and their ability to function.

### DESCRIPTION OF RELATED ART

Platelets are one of the primary components of human blood. Blood is basically made up of plasma, red blood cells (erythrocytes), white blood cells (leukocytes), and platelets (thrombocytes). Platelets are produced in the bone marrow by large cells called megakaryocytes. It is commonly understood that platelets are actually not true cells, but are fragments of membrane and cytoplasm containing granules. More specifically, platelets comprise an outer membrane and cytoplasm from megakaryocytes which in turn contain granules, dense bodies, a dense tubular system, and mitochondria.

It is well recognized that platelets are an essential component of the blood clotting process and play a vital role in controlling bleeding. They adhere specifically to the endothelial cells and the basement membrane lining of damaged blood vessels, where they trigger and participate in hemostasis or clotting. In addition, inflammatory mediators may be released in response to this contact or in response to the mediators released by damaged tissue or other platelets. Important mediators released by platelets include serotonin and coagulation factors. Damaged blood vessels or other vascular breaches are repaired by platelets through such adhesion, and the ensuing response to this type of damage is further amplified by platelet secretions resulting in platelet aggregation and fibrin formation or a stabilized clot.

Platelet transfusions are an important aspect of the clinical management of patients with low numbers of platelets. Normal platelet counts range from about 150,000 to 400,000 per cu/ml. A relatively low number of platelets may be due to cancer treatment and other reasons, or some patients may require transfusions whose platelets are defective in function. Platelets normally aggregate at a site of injury or vessel breakage as described above, and release a number of mediators to which other platelets respond in an amplifying biologic effect or coagulation cascade, which in turn stimulate other biologic effects. The normal, circulating platelet has a disc-shaped morphology. In response to a stimulus, the discs swell into spheres, and may further swell to point where they eventually rupture. Concurrent with this observed change in shape, platelets release a variety of mediators, many of which are released by granules contained within the platelet. The morphology of platelets can be generally determined by microscopic observation. The ability of platelets to maintain their morphology can be tested by subjecting them to mild hypotonic conditions and following their return to disc shape as the membranes pump out excess water. This test is called hypotonic shock response (HSR) and ascertains the ability of the platelet membrane to remain intact during swelling of the platelet and to function by pumping water out of the platelet. Another test of platelet function monitors the change in platelet shape as platelets swell in response to a stimulus. This test is called extent of shape change (ESC).

The process of preparing platelet transfusions typically begins with the separation of platelets as a product from other blood components. Bags of concentrated platelets in blood plasma may be obtained by apheresis or pheresis (centrifugal separation during the donor process while other components are returned to the donor) or by selective removal from whole blood after gravity or centrifugal sedimentation of blood cells. It is very important to preserve platelets after their isolation from the body under suitable conditions that not only maintain the biological activity of the platelets, but also keep them suitable for subsequent clinical use. The average survival time for a platelet in the body after it leaves the bone marrow is eight to ten days. The average expected survival time for circulating platelets is four to five days, which is the average for an entire platelet population. Meanwhile, the current standard and approved method for platelet storage is in a platelet bag that is stored at room temperature for not more than five (5) days. This storage time is limited by the effects of metabolism, including changes in pH, the loss of clinical usefulness, and the risks from growth of small numbers of bacteria that may contaminate the preparation. Some clinicians apply even stricter criteria and decline to use platelets stored for more than three (3) days. The relatively short storage times and the risk of bacterial growth during such storage are major disadvantages and problems associated with current platelet storage methods.

Some platelets in suspension are also stored at reduced temperatures today within normal refrigeration or freezing temperatures ranges. While cold temperature generally serves to suppress bacterial growth, platelets at refrigerator temperatures are known to change shape, lose function, and are cleared from the circulation if transfused. Moreover, at temperatures from about 18° C. to about 14° C., platelet membranes undergo a phase transition that causes membrane defects and adversely effects the platelets. After only a few hours at refrigerator temperature, all discs convert to spheres. Other approaches for preserving platelets have been also reported, including cryopreservation at freezing temperatures in the presence of cryoprotectant such as DMSO. This freezing process is tedious, typically involving gradual lowering of temperature. The recovery of platelets from cryopreservation is also tedious and requires the removal of DMSO and/or other components prior to use in transfusion. Expected platelet recovery from the effects of freezing itself can be relatively low, and the yield is further reduced by subsequent washing in order to remove cryoprotectants or other agents. Satisfactory clinical use has not been reported yet for such platelet preservation techniques.

### SUMMARY OF THE INVENTION

The present invention provides methods and solutions for storage of blood platelets at cold temperatures while substantially maintaining the disc morphology and function of platelets. The refrigerator temperature ranges and cooling methods described herein reduce platelet metabolism and effectively assist in preserving their biological structures and functions, while reducing the risk of bacterial growth during storage. It shall be understood that the particular features and steps for the described embodiments in the specification may be considered individually or in combination with other variations and aspects of the invention.

In one aspect of the invention, the novel methods and solutions herein serve to reduce the adverse effects of membrane phase transition. In yet another aspect of the invention, the novel described methods and solutions serve to reduce the adverse effects of a dual-phase transition (membrane phase transition and cytoplasm phase transition). As a result, these novel methods and solutions can be used for preserving platelets for an extended period of time with reduced loss of biological activity and improved clinical utility.

The collection of platelets for purposes of the invention may be obtained by the usual methods adopted in the industry. The collected platelets to be preserved in accordance with the methods described herein can be suspended in a medium containing one or more polymers such as polyvinlypyrolidone (PVP) or hydroxyethyl starch (HES), and one or more membrane stabilizing agents such as sucrose and magnesium ion. The addition of a polymer assists in stabilizing platelet membranes and reducing the adverse effects of membrane defects that have been known to occur in membrane phase transition.

With respect to one embodiment of the invention, a method is provided for preserving biologically active platelets for storage by transitioning them from a native phase at a temperature above about 18° C. through the transition temperature range to a temperature below 14° C. at a rate exceeding 1° C. per minute, preferably at about 12° C. per minute or faster. Preferably the cooling is continued at a relatively rapid rate to a temperature below 10° C., and optimally to below 6° C. It is a further object of the invention to provide methods of cooling platelet suspensions herein at both uniform and variably adjusted rates of cooling over a predetermined period of time. Platelets may then be stored at a temperature of about −1° C. to about 6° C., preferably at about 0° C. to 4° C. for 1 day or longer, or optionally for more than 3, 5 or 7 days. After a period of storage, the bag of platelets is warmed and used in a transfusion procedure.

In a first example embodiment of the invention, the platelet concentrate was prepared by centrifugation of whole blood and the bag of platelets in plasma was rested on a rocker at 22° C. for 20 hours after which verapamil was added at final concentration 5 mg/L. After 30 minutes, MgCl2 was added to a final concentration of 10 mM. Then 2.25 volumes of a solution containing 2% HES, 2% sucrose, and having an osmolality of about 320 mOsm was added and mixed with the platelet suspension making a final concentration of 1.4% HES and 1.4% sucrose. The bag of platelets was cooled in an ice bath at a rate of 0.2° C. per second to a temperature of 6° C., then held in the ice bath for 10 minutes and transferred to a cold box held at 2° C. After 24 hours in the cold, the bag was warmed in a water bath and tested for numbers of platelets, percent discs, and percent function in assays of hypotonic shock response (HSR) and extent of shape change (ESC). Ten replicates of this experiment were performed, and after 24 hours of storage in the cold, the percentage of original discs remaining ranged from 19% to 93%. After the 24 hours of storage in the cold, the mean values calculated from the 10 replicates were that 93% of the starting platelets were recovered, 44% of starting discs remained as discs, 62% of the original HSR remained, and 14% of the original ESC remained.

In a second example provided by the invention, the platelet concentrate was prepared as in the first example. Verapamil and MgCl2 were added as in the first example, then 2.25 volumes of a solution containing 4.3% PVP (12,000 Da), 2% sucrose, and having an osmolality of about 340 mOsm was added and mixed with the platelet suspension making a final concentration of 3% PVP and 1.4% sucrose. The suspension of platelets was cooled at 0.2° C. per second, stored and warmed as with example one. Again ten replicates were performed, and after 24 hours storage in the cold, 22% to 100% of the original discs remained as discs. After the 24 hours of storage in the cold, the mean of the ten experiments showed 92% of the starting platelets were recovered, 53% of starting discs remained as discs, 79% of the original HSR remained, and 39% of the original ESC remained.

In yet another third example of the invention, the platelet concentrate was also prepared as in the first and second examples, but 2.25 volumes of a solution containing 2% HES, 2% sucrose, 4.3% PVP (12 kDa), and having an osmolality of 360 mOsm was added and mixed with the platelet suspension making a final concentration of 1.4% HES, 1.4% sucrose, and 3% PVP. The suspension of platelets was cooled at 1° C. per second, stored and warmed as in the first and second examples. After 24 hours storage in the cold, 85% of the starting platelets were recovered, 44% of starting discs were recovered as discs, 84% of the original HSR remained, and 27% of the ESC remained.

With respect to a fourth example of the invention, the platelet concentrate was prepared and processed identically to the first example. The platelets were stored at 2° C. for 10 days, warmed according to this invention, and tested. Ten replicates of this experiment were performed. After 10 days storage in the cold the mean values for the ten replicates showed that 78% of the starting platelets were recovered, 20% of the starting discs remained as discs, 33% of the original HSR remained, and 12% of the original ESC remained.

Additionally, a fifth example of the invention includes a platelet concentrate that was also prepared and processed essentially the same as in the first example. The platelet suspension contained 1.4% HES and 1.4% sucrose. The platelet suspension was split into three bags. One bag was placed into a refrigerator and cooled from about 220 C to 20 C at a rate of about 1° C. per minute. Another bag was placed into an ice bath and cooled from about 22° C. to 2° C. at a rate of about 0.05° C. per second. The third bag was placed into an ice bath and cooled at about 0.2° C. per second. All three bags were stored 24 hours at 2° C., and then warmed and tested. No discs survived in the suspension which was cooled at 1° C. per minute. In the suspension that was cooled at about 0.05° C. per second, 40% of the discs remained as discs after 24 hours cold storage and retained 60% of the HSR. In the suspension that was cooled at 0.2° C. per second, 68% of the discs remained after 24 hours storage in the cold and the platelets retained 60% of their HSR.

Another aspect of the invention provides methods of preserving blood platelets without the need for verapamil or magnesium. It shall be understood that alternative embodiments of the invention may include either or both nonetheless. For example, in accordance with this aspect of the invention, example platelets were obtained by apheresis to demonstrate that verapamil and magnesium are not necessarily required for successful cold storage in accordance with the procedures described herein. In another embodiment of the invention, platelets were initially obtained by apheresis using standard methods and a COBE separator. The platelets were then handled according to the procedure set forth in the first example to form platelet suspensions, except that one of the samples did not receive verapamil or magnesium chloride. The refrigerated platelet suspensions were stored for 3 days in the cold and then warmed in a 37° C. water bath and tested. After 3 days of storage at 2° C., 97% of the platelets having added verapamil and MgCl2 were recovered, and had 45% of starting HSR function and 21% of ESC. Meanwhile, 88% of the platelets without added verapamil or MgCl2 were recovered, and had 43% of starting HSR function and 20% of ESC. These and other embodiments or examples of the invention are more fully set forth below in detail.

Other objects and advantages of the invention will become apparent upon further consideration of the specification. While the following description may contain many specific details describing particular embodiments of the invention, this should not be construed as limitations to the scope of the invention, but rather as an exemplification of preferable embodiments. For each aspect of the invention, many variations are possible as suggested herein that are known to those of ordinary skill in the art.

### DETAILED DESCRIPTION OF THE INVENTION

The invention provides methods and solutions for the improved preservation of blood platelets at normal refrigeration temperatures. The disadvantages and limitations of prior preservation methodologies are overcome by the invention which effectively preserves the biological activity of stored platelets under conditions and temperatures achievable with conventional refrigeration apparatus and equipment.

The collection of blood platelets to be preserved and stored may be obtained by usual conventional methods described above. Substances such as theophylline and/or PGE1 may be added to the platelet preparation to get them into quiescence with a high percentage of discs. The suspension of resting platelets is made to contain verapamil (0 to 3 mg/L, optionally 0.3 to 2 mg/L, optionally 1 to 1.5 mg/L), magnesium (Mg) (1 to 10 mM, optionally 1.5 to 3 mM), a carbohydrate such as glucose (0.2% to 4%, optionally 0.5% to 2%) or sucrose (0.2% to 5%, optionally 0.5% to 2%), and a polymer such as hydroxyethyl starch (HES) (0.2% to 5%, optionally 0.5% to 3%, optionally 1% to 2%) or polyvinlypyrrolidone (PVP) (0.5% to 10%, optionally 1% to 6%, optionally 1% to 3%) or dextran (0.5% to 10%, optionally 1% to 6%, optionally 1% to 3%) or any other biocompatible gelling agent that does not activate platelets in a similar range of concentration. The polymers can be used singly or in combination. The osmolarity of the platelet suspension should be normal to slightly hypertonic, at about 300 mOsm to about 380 mOsm. The temperature of the platelet suspension is preferably kept at or above room temperature from about 20° C. to 37° C.

Soon after preparing the described platelet suspension, preferably within an hour, more preferably within 15 minutes, the suspension of platelets is rapidly cooled at a rate faster than about 1° C. per minute, preferably at a rate of about 6° C. per minute, optionally at 12° C. per minute, optionally at 60° C. per minute, optionally at 120° C. per minute (about 2° C per second, optionally faster to about 10° C. per second. The suspension is thus cooled to a temperature below about 10° C., preferably below about 6° C., optionally below about 4° C. to about −6° C. but the formation of ice crystals is minimized.

The cooled suspension of platelets ca be stored at a temperature of about 0° C. to 4° C. and below about 6° C. for a desired duration. Storage can be for up to 1 day, optionally 3 days, optionally 5 days, optionally 7 days, optionally 10 days or longer.

The platelet suspension may be warmed when needed to about 20° C., preferably to about 22° C. or warmer, at a rate of about 2° C. per minute, optionally about 0.1° C. per second or faster. The platelets are then ready for clinical use and may be infused into the blood of a patient.

The benefits of the invention may be demonstrated by counting the intact surviving platelets, and comparing this to the number of platelets before cooling, to provide a measure of the percentage of platelets that are recovered intact and not lost by rupturing or other consequences. The platelets can be assessed for maintaining disc morphology by microscopic observation to ascertain the percentage of platelets that remain as discs compared to having changed into spheres or swollen into balloons. The function and viability of the platelets can be assessed with assays of membrane function to respond to hypotonic conditions (HSR) and shape change in response to an agonist (ESC). If the platelets are sufficiently robust and functional, they can revert from sphere to disc and increase functionality under normal physiologic conditions, for example, after transfusion.

Preferably, up to at least 50%, 60%, 70%, 80% or more of the platelets are recovered from platelet suspensions. In addition, preferably at least 10%, 20%, 30%, 50% or more of the platelets maintain their disc morphology and function for up to at least 2 days, 3 days, 5 days, 7 days, 10 days, and preferably longer in cold storage using the methods and solutions of this invention. Surprisingly, after 24 hours storage in the cold conditions following the procedures described herein, a substantial portion of the platelets remain with natural disc morphology. A substantial portion of the platelets are functional and considered viable. Disc morphology and function are maintained for up to 2 days, 3 days, 5 days, 7 days, 10 days, and longer in cold storage using the methods and solutions of this invention. Preferably, at least 50%, 60%, 70%, 80% or more of the platelets maintain disc morphology and function for at least 2 days, 3 days, 5 days, 7 days, 10 days, and preferably longer in cold storage using the methods and solutions of this invention.

The following non-limiting examples are intended to demonstrate some of the preferable embodiments of the invention. It shall be understood that one skilled in the art will readily recognize that other alternative embodiments may be practiced in order to achieve the effects and benefits of the invention as described herein.

### EXAMPLE 1

### Summary on Stages E(463-472)

In this example, the possibility of saving discoid platelets and their functionality in gelling solutions, containing 1.4% starch, 1.4% of sucrose and 3% of PVP at t+2C by cooling platelets at the rate 0.2 degree C./sec. at t+2C and t−3C under atmospheric pressure, was investigated.

Solutions, Conditions, Test Results

The platelet concentrate (PC) experiments were prepared as follows:

The whole blood collected from one donor was centrifuged at acceleration 1740 g for 4 minutes at t+22C. The platelet rich plasma (PRP) was extracted in a satellite bag and was centrifuged at acceleration 4323 g for 6 minutes at temperature +22C. The supernatant layer of the plasma was excreted, and the volume of suspended plasma with the platelet plaque made 60 ml. The bags with plasma containing platelet plaques were placed on a rocker at t+22C, where they were rested for about 20-22 hours. These bags were assayed and the platelet concentration in the PC was determined. Then the assay taken from the PC (Test 1) was tested. The bags were added the verapamil solution so that the final concentration made 5 mg/L. The bags were placed on the rocker at t+22C where they were rested for 30 minutes. Then the PC was added the solution MgCl2 so that its final concentration made 0.95 g/L (10 mM). Then each bag with the PC collected from the one donor was split into 3 sets of bags.

The bags with Numbers 1 were added 2.25 volumes of an additive solution I containing HES—2% and sucrose—2% in the solution “Transvect” (osmolality 320 to 325 mOsm/L and pH 7.53 to 7.55). In view of dilution of these bags the concentration of HES made 1.4% and that of sucrose 1.4%. The osmolality of the obtained PC solution made about 322 to 330 mOsm/L.

The bags with Numbers 2 were added 2.25 volumes of an additive solution II containing PVP—4.3% and sucrose—2% in the solution “Transvect” (osmolality 343 to 348 mOsm/L and pH 7.47 to 7.5). In view of dilution of these bags the concentration of PVP made 3% and that of sucrose 1.4%. The osmolality of the obtained solution made about 340 to 346 mOsm/L.

The bags with Numbers 3 were added 2.25 volumes of an additive solution II containing PVP—4.3% and sucrose—2% in the solution “Transvect” (osmolality 343 to 348 mOsm/L, and pH 7.47 to 7.5). In view of dilution of these bags the concentration of PVP made 3% and that of sucrose 1.4%. The osmolality of the obtained solution made about 341 to 346 mOsm/L.

The final concentration of inhibitors made: verapamil—1.55 mg/L; MgCl2—0.29 g/L (3.1 mM).

The bags 1 and 2 were cooled in the ice bath at t+18C down to t+6C at the rate 0.2 degrees C./sec. These bags were held in the ice bath for 10 minutes and then were transferred to the incubator at t+2C.

The bags 3 were cooled from t+18C down to t+6C at the rate 0.17 to 0.23 degrees C./sec. (the average rate is 0.21 degrees C./sec.) in a saline solution at t−4C. The time of cooling down to t−3C was 4 to 5 minutes. These bags were held for 20 minutes at t−3C and transferred into the incubator at t+2C. Note that the bags 1, 2 and 3 during cooling were kept in a stabile condition, and the cooling liquid was agitated around the bags.

After storage for 24 hours, the bags with Numbers 1, 2 and 3 were warmed up t+22C in the water bath and assayed for testing: Test 2 for the bags with Numbers 1, Test 4 for the bags with Numbers 2, and Test 6 for the bags with Numbers 3. HSR and ESC were not analyzed in these Tests. The assays with the solutions taken from bags were placed on a bay at a room temperature (about t+25C to 27C) where they were rested for 3-4 hours before the percent of the platelet shape. During this time, the following assay testing with solutions was made including definition of the platelet concentration and osmolality and pH of solutions, and also testing of the washed out platelet-HSR and ESC, except for the already specified parameters. The assays taken from bags, were centrifuged at acceleration 1460 g within 10 minutes.

The supernatant layer of plasma was excreted and the volume was adjusted to the stock with the donor plasma. The PRP assays were placed on the rocker at temperature +22C where they were rested for 1 hour. Then these assays were tested: Test 3 for bags with Numbers 1, Test 5 for bags with Numbers 2, and Test 7 for bags with Numbers 3.

Per day of a loading platelet for storage an additive solution I containing HES—2% and sucrose—2% was prepared and additive solution II, containing PVP—4.3% and sucrose—2%, in a saline solution “Transvect.” For this purpose 2 g of sucrose and 2 g of HES were dissolved in 92.5 ml of distilled water as follows. In one half of the water volume required for the preparation of the given HES solution, the specified amounts of sucrose and HES were added and the obtained suspension was carefully agitated. The second half of the required water volume was warmed up in a water bath to t+100C. This hot water was gradually added a solution containing a suspension of HES at continuous stirring during 10 to 20 minutes before complete dissolution of HES. In 92.5 ml of distilled water there were dissolved 2 g of sucrose and 40.3 g of PVP at ambient temperature.

The concentrated saline solution “Transvect” was previously prepared. Each salt: KCl—2.24 g (30 mmol), K2HPO4—14.8 g (85 mmol), KH2PO4—4.1 g (30 mmol) and NaHCO3—1.68 g (20 mmol), was dissolved in 20 ml of distilled water separately at ambient temperature. Then the obtained solutions of salts were admixed also their general volume was brought to 100 ml. In general solution of salt the content made: KCl—0.30 mmol/mL, K2HPO4—0.85 mmol/mL, KH2PO4—0.30 mmol/mL and NaHCO3—0.20 mmol/mL. From this solution, 7.5 ml containing KCl—2.25 mmol, K2HPO4—6.38 mmol, KH2PO4—2.25 mmol and NaHCO3—1.5 mmol, were taken and added in 92.5 ml of additive solutions I and II. As a result of concentration of ions in these additive solutions made: K⁺—173 mM; Na⁺—15 mM; HPO4—86 mM; HCO3—15 mM. Then 20 ml of the PC, containing verapamil and MgCl2 (10 mM), were each added 45 ml of the additive solutions containing the specified ions, i.e. at such dilution the concentration of the added ions reduced to 0.69 from their concentration in the stock additive solutions. In a final solution containing platelets, the concentration of ions, assuming the plasma ions, made: K⁺—121 mM; Na⁺—55 mM; Mg²⁺—3.4 mM; Cl⁻—38 mM; HPO₄⁻—60 mM; HCO₃⁻—13.4 mM; SO₄²⁻—0.06 mM. Thus, in a final solution the concentration of the added ions were close to their concentration inside cells, though in the solution the concentration of Na and Cl ions considerably exceeded them.

The microscopy of the assays taken from bags with Numbers 1 demonstrated that at a room temperature the gel in solutions survived in the solutions though in the assays taken from the bags with Numbers 2 and 3, no gel was found, however the solutions were viscous. Therefore, it was difficult to determine the rate of disks in such solution with high viscosity. The fragments, single dendrites, small platelet aggregates and altered disks were found in these assays. The discoid platelets were oblong and convex, many of them had one pseudopodium each.

After centrifugation and washing out of the platelet, microfragments and small platelet aggregates were found almost in every assay.

In the ANALYSIS supplement immediately below we provide a statistical analysis of received results on the platelet concentration, osmolality and pH of solutions, percents of disks, HSR and ESC and optical densities Dₒ.

The average values of the platelet of parameters expressed in percentage in relation to these parameters in the stock PRP are submitted in the Summary Table. And the stock platelet concentrations in all bags were so that after getting in 2.25 volumes of an additive solution in PC the platelet count depressed up to 0.31.

In the Summary Table, the average value of the platelet count, percents of disks, HSR and ESC in all bags after storage, are given at temperature +2C. Confidence intervals appropriate to confidence probability are specified there 0.95. Thus, a significance level of the received parameters p≦0.05, i.e. with the probability 0.05 the measured parameters can fall outside the range of confidence intervals.

Summary Table: E(463-472), p ≦ 0.05.

| | Concentration, | Discs, | HSR, | ESC, |
|---|---|---|---|---|
| | % | % | % | % |
| Bag 1: | 93 ± 14.7 | 44 ± 53.1 | 62 ± 61 | 14 ± 31.7 |
| starch-1.4%, | | | | |
| sucrose-1.4%. | | | | |
| Bag 2: | 92 ± 15.7 | 53 ± 66.5 | 79 ± 72 | 39 ± 40 |
| PVP-3%, | | | | |
| Sucrose-1.4% | | | | |
| Bag 3: | 93 ± 17 | 53 ± 67.8 | 75 ± 67 | 26 ± 36.9 |
| PVP-3%, | | | | |
| Sucrose-1.4% | | | | |

As it is visible from the given data, the platelet parameters alter within wide ranges. Perhaps, it is a consequence of individual properties of the donor's PC that which are caused by composition of plasma, frame and defects of membranes of thrombocytes. Probably, the safety and the platelet function are affected by additive solution and the rate of cooling of the PC solutions, which varies within some limits. An average quadratic deviation σRe, determining the borders of a confidence interval is in inverse proportion (n−1)^{1/2} where n—number of experiments. Therefore to reduce a confidence interval it is necessary to carry out many experiments (n>10).

Based on the results of the described experiments it follows, that at presence of PVP the disks survive, though in a solution with PVP a soft gel generates. Apparently, the molecules of PVP protect the platelet membranes during fast cooling and, partially keeping free water in a solution, interfere with the platelet swelling. Probably, the molecules of sucrose, when penetrating through transmembrane defects raise the osmolality of the cytoplasma and promote the swelling of the platelets.

After storage of function of the platelets is determined on HSR and ESC, were depressed in PVP solution. Probably, the factor causing loss of functions by the platelets was the redistribution outside of and endocellular ions because of gradients of their concentration. Another factor, owing to which HSR declined, was probably the partial swelling of the cytoplasma causing transmembrane defects, in a PVP solution, forming a soft gel. In the cytoplasma of the platelets in bags with Numbers 3 and cooled down to t−3C, more viscid gel was generated than in the cytoplasma of the platelets in bags with Numbers 2 and cooled down to t+2C.

Therefore in bags with Numbers 3, a degree of swelling was greater than in bags with Numbers 2.

In result in bags with Numbers 3, HSR was lower than in bags with Numbers 2. Thus, cooling and holding platelet at subzero temperatures probably accelerates transferring of the cytoplasma in gel phase and promotes survival of discs and the platelet functionality though under these conditions a gel of definite viscosity should be in a solution. As the rate of disks was determined after holding the PC at temperatures +25 to +27 degrees during 3 to 4 hours, a definite conclusion is not possible about the safety of disks at temperature +2C. It is quite probable that a part of spherical platelets stored at temperature +2C, transformed into disks under the specified conditions, in spite of the fact that the solutions the containing ions in concentration close to their endocellular somewhat inhibit a metabolism.

### Analysis

Test Results of the Stages E463-E472 Experiments made Under Invariable Conditions.

1. Processing of the Test Results.

1.1. Processing of the results made on relative indexes are given in relation to the parameters of Test T1. The values of the parameters under the Test T1 are accepted for 100%. The value of relative indexes under the Tests T2 to T7, specified in the Tables below, were determined under the general formula:

Ri=100PTi:PT1,

where Ri—value of a relative index on Test “i”, %; PTi—absolute value of parameter on Test “i”; PT1—absolute value of parameter under the Test T1.

1.2. Mathematical expectation (average arithmetic) MRe and average quadratic deviation σRe were determined under the formulas:

MRe=1/nΣRe; σRe=[1/(n−1)Σ(Re−MRe)²]^{1/2},

where Re—value of a relative index on Test “e” experiment.

1.3. Estimation of confidence intervals: the bottom value R_{b} and top Rₜ—was made with confidence probability 0.95 (quantile Up=1,985).

2. Processing of the Test Results.

2.1. The results of definition of relative indexes of parameter PI.Concentration are specified in the Table 1.

TABLE 1

| Experiment | T1 | T2 | T3 | T4 | T5 | T6 | T7 |
|---|---|---|---|---|---|---|---|
| E463 | 100 | 103 | 94 | 99 | 80 | 101 | 87 |
| E464 | 100 | 94 | 54 | 101 | 77 | 103 | 68 |
| E465 | 100 | 101 | 55 | 99 | 69 | 103 | 81 |
| E466 | 100 | 97 | 73 | 95 | 84 | 92 | 82 |
| E467 | 100 | 100 | 59 | 96 | 47 | 101 | 58 |
| E468 | 100 | 89 | 58 | 86 | 32 | 93 | 35 |
| E469 | 100 | 87 | 54 | 84 | 79 | 84 | 79 |
| E470 | 100 | 90 | 88 | 76 | 72 | 88 | 87 |
| E471 | 100 | 79 | 53 | 90 | 63 | 81 | 77 |
| E472 | 100 | 90 | 54 | 90 | 102 | 84 | 80 |
| MPI.C | — | 93 | 64 | 92 | 71 | 93 | 73 |
| σPI.C | — | 7.4 | 15.3 | 7.9 | 19.6 | 8.6 | 16.1 |
| σPI.C*Up | — | 14.7 | 30.5 | 15.7 | 38.9 | 17 | 31.9 |
| PI.Cb | — | 78.3 | 33.5 | 76.3 | 32.1 | 76 | 41.1 |
| PI.Ct | — | 107.7 | 94.5 | 107.7 | 109.9 | 110 | 104.9 |

2.2. The results of definition of relative indexes of parameter of the Osmolality are specified in the Table 2.

TABLE 2

| Experiment | T1 | T2 | T3 | T4 | T5 | T6 | T7 |
|---|---|---|---|---|---|---|---|
| E463 | 100 | 107 | 101 | 113 | 101 | 113 | 100 |
| E464 | 100 | 105 | 101 | 109 | 100 | 110 | 99 |
| E465 | 100 | 102 | 99 | 107 | 98 | 107 | 97 |
| E466 | 100 | 104 | 100 | 110 | 98 | 110 | 99 |
| E467 | 100 | 103 | 102 | 108 | 102 | 108 | 103 |
| E468 | 100 | 106 | 103 | 111 | — | 112 | — |
| E469 | 100 | 104 | 100 | 109 | 100 | 109 | 100 |
| E470 | 100 | 105 | 100 | 110 | 100 | 110 | 101 |
| E471 | 100 | 108 | 102 | 111 | 102 | 112 | 103 |
| E472 | 100 | 105 | 102 | 110 | 101 | 110 | 101 |
| MOsm | — | 105 | 101 | 110 | 100 | 110 | 100 |
| σOsm | — | 1.8 | 1.2 | 1.7 | 1.5 | 1.9 | 1.9 |
| σOsm*Up | — | 3.6 | 2.5 | 3.3 | 2.9 | 3.7 | 3.8 |
| Osm b | — | 101.4 | 98.5 | 106.7 | 97.1 | 106.3 | 96.2 |
| Osm t | — | 108.6 | 103.5 | 113.3 | 102.9 | 113.7 | 103.8 |

2.3. The results of definition of relative indexes of parameter of the pH are specified in the Table 3.

TABLE 3

| Experiment | T1 | T2 | T3 | T4 | T5 | T6 | T7 |
|---|---|---|---|---|---|---|---|
| E463 | 100 | 109 | 109 | 108 | 108 | 108 | 108 |
| E464 | 100 | 112 | 113 | 112 | 112 | 111 | 112 |
| E465 | 100 | 109 | 109 | 108 | 108 | 108 | 108 |
| E466 | 100 | 113 | 112 | 111 | 112 | 110 | 112 |
| E467 | 100 | 103 | 104 | 102 | 103 | 102 | 103 |
| E468 | 100 | 106 | 106 | 104 | — | 104 | — |
| E469 | 100 | 106 | 107 | 105 | 106 | 104 | 106 |
| E470 | 100 | 105 | 105 | 103 | 104 | 103 | 103 |
| E471 | 100 | 106 | 106 | 104 | 105 | 104 | 105 |
| E472 | 100 | 103 | 103 | 102 | 103 | 102 | 102 |
| MpH | — | 107 | 107 | 106 | 107 | 106 | 107 |
| σpH | — | 3.5 | 3.3 | 3.6 | 3.5 | 3.3 | 3.7 |
| σpH*Up | — | 6.9 | 6.6 | 7.2 | 6.9 | 6.6 | 7.4 |
| pHb | — | 100.1 | 100.4 | 98.8 | 100.1 | 99.4 | 99.4 |
| pHt | — | 113.9 | 113.6 | 113.2 | 113.9 | 112.6 | 114.4 |

2.4. The results of definition of relative indexes of parameter of Discs are specified in the Table 4.

TABLE 4

| Experiment | T1 | T2 | T3 | T4 | T5 | T6 | T7 |
|---|---|---|---|---|---|---|---|
| E463 | 100 | 19 | single | 24 | single | 21 | single |
| E464 | 100 | 22 | single | 31 | single | 32 | single |
| E465 | 100 | 23 | single | 22 | single | 25 | single |
| E466 | 100 | 22 | single | 24 | single | 18 | single |
| E467 | 100 | 97 | single | 130 | single | 130 | single |
| E468 | 100 | 28 | single | 48 | single | 46 | single |
| E469 | 100 | 57 | single | 48 | single | 57 | single |
| E470 | 100 | 36 | single | 57 | single | 50 | single |
| E471 | 100 | 69 | single | 69 | single | 67 | single |
| E472 | 100 | 66 | single | 80 | single | 82 | single |
| MDiscs | — | 44 | | 53 | | 53 | |
| σDiscs | — | 26.8 | | 33.5 | | 34.2 | |
| σDiscs*Up | — | 53.1 | | 66.5 | | 67.8 | |
| Discs b | — | −9.1 | | −13.5 | | −14.8 | |
| Discs t | — | 97.1 | | 119.5 | | 120.8 | |

2.5. The results of definition of relative indexes of parameter of Dendrites are specified in the Table 5.

TABLE 5

| Experiment | T1 | T2 | T3 | T4 | T5 | T6 | T7 |
|---|---|---|---|---|---|---|---|
| E463 | 100 | 33 | single | 100 | single | 133 | single |
| E464 | 100 | 100 | single | 167 | single | 133 | single |
| E465 | 100 | 100 | single | 133 | single | 133 | single |
| E466 | 100 | 75 | single | 50 | single | 100 | single |
| E467 | 100 | 67 | single | 67 | single | 117 | single |
| E468 | 100 | 125 | single | 200 | — | 150 | — |
| E469 | 100 | 33 | single | 60 | single | 83 | single |
| E470 | 100 | 40 | single | 100 | single | 60 | single |
| E471 | 100 | 125 | single | 75 | single | 100 | single |
| E472 | 100 | 125 | single | 125 | single | 75 | single |
| MDend | — | 82 | | 108 | | 108 | |
| σDend | — | 38 | | 48.8 | | 29.6 | |
| σDend*Up | — | 75.5 | | 96.8 | | 58.7 | |
| Dend b | — | 6.5 | | 11.2 | | 49.3 | |
| Dend t | — | 157.5 | | 204.8 | | 166.7 | |

2.6. The results of definition of relative indexes of parameter of the HSR are specified in the Table 6.

TABLE 6

| Experiment | T1 | T2 | T3 | T4 | T5 | T6 | T7 |
|---|---|---|---|---|---|---|---|
| E463 | 100 | — | 61 | — | 40 | — | 48 |
| E464 | 100 | — | 74 | — | 37 | — | 43 |
| E465 | 100 | — | 44 | — | 125 | — | 39 |
| E466 | 100 | — | 48 | — | 61 | — | 85 |
| E467 | 100 | — | 49 | — | 42 | — | 73 |
| E468 | 100 | — | 22 | — | — | — | — |
| E469 | 100 | — | 135 | — | 76 | — | 57 |
| E470 | 100 | — | 68 | — | 91 | — | 107 |
| E471 | 100 | — | 75 | — | 112 | — | 143 |
| E472 | 100 | — | 39 | — | 126 | — | 77 |
| MHSR | — | | 62 | | 79 | | 75 |
| σHSR | — | | 30.7 | | 36.3 | | 33.8 |
| σHSR*Up | — | | 61 | | 72 | | 67 |
| HSR b | — | | 1 | | 7 | | 8 |
| HSR t | — | | 123 | | 151 | | 142 |

2.7. The results of definition of relative indexes of parameter of the ESC are specified in the Table 7.

TABLE 7

| Experiment | T1 | T2 | T3 | T4 | T5 | T6 | T7 |
|---|---|---|---|---|---|---|---|
| E463 | 100 | — | 18 | — | 45 | — | 27 |
| E464 | 100 | — | 27 | — | 40 | — | 40 |
| E465 | 100 | — | 19 | — | 25 | — | 0 |
| E466 | 100 | — | 17 | — | 22 | — | 28 |
| E467 | 100 | — | 0 | — | 56 | — | 50 |
| E468 | 100 | — | 50 | — | — | — | — |
| E469 | 100 | — | 0 | — | 50 | — | 50 |
| E470 | 100 | — | 0 | — | 67 | — | 0 |
| E471 | 100 | — | 13 | — | 44 | — | 19 |
| E472 | 100 | — | 0 | — | 0 | — | 21 |
| MESC | — | | 14 | | 39 | | 26 |
| σESC | — | | 16 | | 20.2 | | 18.6 |
| σESC*Up | — | | 31.7 | | 40 | | 36.9 |
| ESC b | — | | −17.7 | | −1 | | −19.9 |
| ESC t | — | | 45.7 | | 79 | | 62.9 |

2.8. The results of definition of relative indexes of parameter of the optical density Dₒ are specified in the Table 8.

TABLE 8

| Experiment | T1 | T2 | T3 | T4 | T5 | T6 | T7 |
|---|---|---|---|---|---|---|---|
| E463 | 100 | — | 117 | — | 106 | — | 76 |
| E464 | 100 | — | 156 | — | 119 | — | 116 |
| E465 | 100 | — | 114 | — | 105 | — | 115 |
| E466 | 100 | — | 106 | — | 106 | — | 98 |
| E467 | 100 | — | 99 | — | 104 | — | 80 |
| E468 | 100 | — | 100 | — | — | — | — |
| E469 | 100 | — | 90 | — | 96 | — | 85 |
| E470 | 100 | — | 103 | — | 104 | — | 89 |
| E471 | 100 | — | 126 | — | 126 | — | 109 |
| E472 | 100 | — | 121 | — | 71 | — | 92 |
| MDₒ | — | | 113 | | 104 | | 96 |
| σDₒ | — | | 18.7 | | 15.3 | | 14.9 |
| σDₒ*Up | — | | 37 | | 30.4 | | 29.6 |
| Dₒ b | — | | 76 | | 73.6 | | 66.4 |
| Dₒ t | — | | 150 | | 134.4 | | 125.6 |

### EXAMPLE 2

### Summary on Stages E(508-509), E(512-513), E(516-517), E(519-524)

In this example, the possibility of saving discoid platelets and their functionality in gelling solutions, containing starch—1.4%, sucrose—1.4% and PVP—3% at t+2C by cooling platelets at the rate 0.2 degree C./sec. at t+2C and t−3C under atmospheric pressure, was investigated.

Solutions, Conditions, Test Results

The PC for experiments was prepared as follows:

The whole blood collected from one donor was centrifuged at acceleration 1740 g for 4 minutes at t+22C. The PRP was extracted in a satellite bag and was centrifuged at acceleration 4323 g for 6 minutes at temperature +22C. The supernatant layer of the plasma was excreted and the volume of suspended plasma with the platelet plaque made 60 ml. The bags with plasma containing platelet plaques were placed on the rocker at t+22C, where they were rested for 20 to 22 hours. These bags were assayed and the platelet concentration in the PC was identified. Then the assay taken from the PC (Test 1) was tested. The bags were added the verapamil solution, so that the final concentration made 5 mg/L. The bags were placed on the rocker at t+22C, where they were rested for 30 minutes. Then the PC was added the solution MgCl2 so that its final concentration made 0.95 g/L (10 mM). Then each bag with the PC collected from one donor was split into 3 sets of bags.

The bags with Numbers 1 were added 2.25 volumes of an additive solution I containing HES—2% and sucrose—2% in the solution “Transvect” (osmolality 320 to 325 mOsm/L and pH 7.53 to 7.55). In view of dilution of these bags the concentration of HES made 1.4% and that of sucrose 1.4%. The osmolality of the PC solution made 324 to 334 mOsm/L.

The bags with Numbers 2 were added 2.25 volumes of an additive solution II containing PVP—4.3% and sucrose—2% in the solution “Transvect” (osmolality 343 to 348 mOsm/L and pH 7.47 to 7.5). In view of dilution of these bags the concentration of PVP made 3% and that of sucrose 1.4%. The osmolality of the obtained solution made 340 to 343 mOsm/L.

The bags with Numbers 3 were added 2.25 volumes of an additive solution II containing PVP—4.3% and sucrose—2% in the solution “Transvect” (osmolality 343 to 348 mOsm/L, pH 7.47 to 7.5). In view of dilution of these bags the concentration of PVP made 3% and that of sucrose 1.4%. The osmolality of the obtained solution made 340 to 343 mOsm/L.

The final concentration of inhibitors made: verapamil—1.55 mg/L; MgCl2—0.29 g/L (3.1 mM).

The bags 1 and 2 were cooled in the ice bath from t+18C down to t+6C at the average rate 0.2 degrees C./sec. These bags were held in the ice bath for 10 minutes and then were transferred to the incubator at t+2C.

The bags 3 were cooled from t+18C down to t+6C at the rate 0.17 to 0.23 degrees C./sec. (the average rate is 0.21 degrees C./sec.) in a saline solution at t−4C. The time of cooling down to t−3C was 4 to 5 minutes. These bags were held for 20 minutes at t−3C and transferred in to the incubator at t+2C. Note that the bags 1, 2, 3 during cooling were kept in a stabile condition and the cooling liquid was agitated around the bags.

After storage for 240 hours, the bags with Numbers 1, 2 and 3 were warmed up to t+22C in the water bath and assayed for testing: Test 2 for the bags with Numbers 1, Test 4 for the bags with Numbers 2, and Test 6 for the bags with Numbers 3. HSR and ESC were not analyzed in these Tests. The assays with the solutions taken from bags, were placed on a bay at a room temperature (t+26C to 28C) where they were rested for 3-4 hours before the percent of the platelet shape was determined. During this time the assay testing with solutions was made: definition of the platelet concentration and osmolality and pH of solutions, and also testing of the washed out platelet-HSR and ESC, except for the already specified parameters. The assays taken from bags, were centrifuged at acceleration 1460 g for 10 minutes.

The supernatant layer of plasma was excreted and the volume was brought in the stock volume with the donor plasma. The PRP assays were placed on the rocker at temperature +22C where they were rested for 1 hour. Then these assays were tested: Test 3 for bags with Numbers 1, Test 5 for bags with Numbers 2, and Test 7 for bags with Numbers 3.

On the day of loading platelet for storage, an additive solution I containing HES—2% and sucrose—2% was prepared and additive solution II, containing PVP—4.3% and sucrose—2%, in a saline solution “Transvect.” For this purpose 2 g of sucrose and 2 g of HES were dissolved in 92.5 ml of distilled water as follows. In one half of water volume required for the preparation of the given HES solution, the specified amounts of sucrose and HES were added and the obtained suspension was thoroughly mixed. The second half of the required water volume was warmed up in a water bath to t+100C. This hot water was gradually added a solution containing a suspension of HES at continuous stirring during 10 to 20 minutes before complete dissolution of HES. In 92.5 ml of distilled water there were dissolved 2 g of sucrose and 4.3 g of PVP at ambient temperature.

The concentrated saline solution “Transvect” was previously prepared. Each salt: KCl—2.24 g (30 mmol), K2HPO4—14.8 g (85 mmol), KH2PO4—4.1 g (30 mmol) and NaHCO3—1.68 g (20 mmol) was dissolved in 20 ml of distilled water separately at ambient temperature. Then the obtained solutions of salts were admixed also their general volume was brought to 100 ml. In a general solution of salt the content made: KCl—0.30 mmol/ml, K2HPO4—0.85 mmol/ml, KH2PO4—0.30 mmol/ml and NaHCO3—0.20 mmol/ml. From this solution 7.5 ml containing KCl—2.25 mmol, K2HPO4—6.38 mmol, KH2PO4—2.25 mmol and NaHCO3—1.5 mmol were taken and added in 92.5 ml of additive solutions I and II. As a result of concentration of ions, in these additive solutions made: K⁺—173 mM; Na⁺—15 mM; Cl⁻—22.5 mM; HPO4—86 mM; HCO3—15 mM. Then 20 ml of the PC, containing verapamil and MgCl2 (10 mM), were each added 45 ml of the additive solutions containing the specified ions, i.e. at such dilution the concentration of the added ions reduced to 0.69 from their concentration in the stock additive solutions. In a final solution containing platelets, the concentration of ions, assuming the plasma ions, made: K⁺—121 mM; Na⁺—55 mM; Mg²⁺—3.4 mM; Cl⁻—54.2 mM; HPO₄⁻—60 mM; HCO₃⁻—13.4 mM; SO₄²⁻—0.06 mM. Thus, in a final solution the concentration of the added ions were close to their concentration inside cells, though in the solution the concentration of Na and Cl ions considerably exceeded them.

The microscopy of the assays taken from bags with Numbers 1 demonstrated that at a room temperature the gel survived in the solutions though in the assays taken from the bags with Numbers 2 and 3, no gel was found, however the solutions were viscous. Therefore, it was difficult to analyze disks in such solution with high viscosity. The microfragments, single dendrites, small platelet aggregates and altered disks were found in these assays. Moreover, in the assays taken from the bags with Numbers 2 and 3 no gel was found, though the solutions were viscid.

In the assays single microfragments, single platelet aggregates and discoid platelets were found. The discoid platelets have both perfect and altered shape. The relation between perfect and altered discs were about 1:4. The altered discs were oblong and convex, many of them had one pseudopodium each. Besides, in the assays from the bags 2 and 3 some single microfragments, single platelet aggregates and conglutinated platelets. Besides in many assays taken from the bags the balloons were also found out. After centrifugation and washing out of the platelet, microfragments and small platelet aggregates were found almost in every assay.

In a Stage E519, the following experiments were carried out. One assay taken from bag 1 upon storage at t+2C, at first was warmed up to t+37C in a water bath, and then was rested in the incubator at temperature +37C within 30 minutes. Then rate of disks in this assay was determined. As it was found out subsequently, this rate of disks was higher, than in then that one in the assay taken from bag 1 and stored at ambient temperature within 3 hours. After storing the assays taken from bags 1, 2 and 3, at temperature +28C within 3 hours the platelet morphology was determined and the RI was filled in with these data. In about an hour, the platelet morphology was determined repeatedly in these assays which had been held at the specified temperature. The rate of disks had essentially increased. This data demonstrated that after storage at +2C and warming up, a sphere-to-disc transformation occurred in the assays at an ambient temperature, as well as at +37C. It is necessary to note that the platelets transformed in a solution containing ions not in a physiological concentration.

In the ANALYSIS Supplement immediately below we are providing a statistical analysis of data obtained on the platelet concentration, osmolality and pH of solutions, percents of disks, HSR and ESC and optical densities Dₒ.

The average values of the platelet of parameters expressed in percentage in relation to these parameters in the stock PRP are submitted in the Table. And the stock platelet concentrations in all bags were so that after getting in 2.25 volumes of an additive solution in PC the platelet count depressed up to 0.31.

In the Summary Table the average value of the platelet count, percents of disks, HSR and ESC in all bags after storage are given at temperature +2C. Confidence intervals appropriate to confidence probability are specified there 0.95. Thus, a significance level of the received parameters p≦0.05, i.e. with the probability 0.05 the measured parameters can fall outside the range of confidence intervals.

Summary Table:
E(508-509), E(512-513), E(516-517), E(519-524); p ≦ 0.05.

| | Concentration, | Discs, | HSR, | ESC, |
|---|---|---|---|---|
| | % | % | % | % |
| Bag 1: | 78 ± 30.1 | 20 ± 21.5 | 33 ± 60.8 | 12 ± 22.7 |
| Starch-1.4%, | | | | |
| Sucrose-1.4%. | | | | |
| Bag 2: | 65 ± 31.6 | 12 ± 18.7 | 22 ± 44.0 | 7 ± 25.9 |
| PVP-3%, | | | | |
| Sucrose-1.4% | | | | |
| Bag 3: | 68 ± 26.4 | 9 ± 12.0 | 25 ± 30.7 | 7 ± 23.8 |
| PVP-3%, | | | | |
| Sucrose-1.4% | | | | |

In FIGS. 1, 2 and 3, the temporary dependences of the average parameters (platelet count, percent of disks, HSR and ESC), obtained for bags 1, 2, 3, respectively, are given: 24 hours—E (463-472); 48 hours—E (473-482); 120 hours—E (493-502); 168 hours—E (503-507); E (510-511); E(514-515); E518; 240 hours—E(508-509), E (512-513), E (516-517), E (519-524).

[image]

[image]

[image]

As it is visible from the given data, the platelet parameters of thrombocytes change in wide ranges. Apparently, it is a consequence of individual properties of the PC donors, which are caused by composition of plasma, frame and defects of membranes of the platelets. An additive solution and rate of cooling of the PC solutions may affect the platelet safety and functions, which varies within some range.

Based on the results of the described experiments it follows, that at a presence of PVP the disks survive, though in a PVP solution a weak gel form. Apparently, the molecules PVP protect the platelet membranes during fast cooling and, partially keeping free water in a solution, interfere the platelet swelling. The molecules of sucrose, penetrating through transmembrane defects, may raise the osmolality of the cytoplasma and promote the platelet swelling. After storage, the platelet function determined by HSR and ESC values were depressed in PVP solution. The factor causing loss of the platelet functions appeared to be a redistribution of extracellular and endocellular ions because of gradients of their concentration. As a result, the concentration of ions of Na and Cl raised during shelf-life at temperature +2C. After warming up to ambient temperature and disintegration of gel in the PC solution, a swelling of the platelets occurred accompanied by an excretion of amino acids from cells. Another factor, owing to which HSR has dropped, may be due to the partial swelling of the cytoplasma causing occurrence of transmembrane defects, in PVP solution, forming a weak gel. And in the cytoplasma of the platelets, being in bags with Numbers 3 and cooled down to t−3C, more viscid gel was formed, than in the cytoplasma of the platelets being in bags with Numbers 2 and cooled down to t+2C. Thus, cooling down and the endurance of the platelets at subzero temperatures, may promote transferring the cytoplasma in a phase of gel and promote disks and platelet functions survival but under these conditions in a solution a gel with a definite viscosity should be present.

The results on storing the PC solutions demonstrate that the most significant changes of the platelet parameters occur during cooling-off period and storage within 24 hours at temperature t+2C. And in a solution containing HES, the drop of the platelet parameters is more, than in a solution containing PVP, in this period of time. At further storage the average parameters of the platelets in both solutions are averaged. From here it follows, that at a fast cooling PVP protects the platelet membranes to a greater degree than HES. In view of that, in a solution containing PVP, less viscid gel is formed, than in a solution containing HES, in the first solution the degree of the platelets swelling is more, than in the second solution. As a result, the rate of decreasing of the platelet parameters in a solution containing PVP is greater. The other factor causing dropping of the platelet functions is the accumulation of intermediate products of biochemical reactions in cells during long-term storage of the platelets at temperature +2C. On the one hand, these intermediate products having high osmolality and promote the platelet swelling. And, on the other hand, rising of their concentration results in inhibition of metabolism and dropping of HSR and ESC of the platelets after storage. Also it is possible, that the platelet functions are depressed owing to small gradients of concentration of the extracellular and endocellular ions and decrease of absolute sizes of membrane potentials. During shelf-life of the platelets from 24 till 48 hours the rise of the percents of disks and ESC in the assays taken from bags 2 and 3 and containing PVP was observed. It specifies that during this period of time, a healing of transmembrane defects occurred during cooling platelets. If in the described conditions the integrity of membranes is substantially preserved, we could expect a reduction of the disk shape and functions of the platelets in vivo after their infusion and circulation in a blood channel.

It is necessary to note that ESC values, which was determined after centrifugation and the washing out platelets, were not equal to zero in the assays taken from all bags. In these assays however only the single disks were determined. There may be at least two reasons for such phenomenon: first, in the assays there was an appreciable amount of non-identified discs; and secondly, getting in ADP in PRP had induced the platelet activation, accompanied by a reaction of release and occurrence of microparticles, that has declined PRP transmission coefficient. As it was already specified above, in the assays taken from bags with Numbers 2 and 3, single microparticles were observed.

On oblique acknowledgement of the low amount of microparticles serve the dropping of optical densities of the PC solution in the bags with Numbers 2 and 3 after storage at t+2C. The transformation of the platelets is accompanied by reaction of release and vesiculation of their membranes. Therefore a small amount of microparticles in the PC is the oblique certificate only of partial disc-to-sphere transformation of the platelets after cooling and storage in solutions containing PVP and sucrose.

As it was specified earlier, after fast warming up to t+22C in the PC solutions, the gel survived in which it is difficult to identify the platelets count. Therefore, the assays of the PC solutions were held at temperature +25C to +28C, at which there was a disintegration of gel within 3-4 hours. Perhaps, during this period of time both disc-to-sphere and reverse transformation of the platelet occurred. From here it follows, that for exact analysis of rate of discoid platelets it is necessary to fix their shapes either at temperature +2C, or at ambient temperature upon warming up with the help of special chemical solutions. This need is urgent, as the rate of the discs survived is a major parameter, on which the efficiency of additive solutions and conditions of storage of the platelets is determined. Thus, for proceeding with optimization of the compositions of additive solutions, the rates of cooling and platelet storage conditions first of all a technique of fixing platelet and analyze actual rate of the discoid platelets survived.

### Analysis

### Test Results of the Stages E(508-509), E(512-513), E(516-517), E(519-524) Experiments made Under Invariable Conditions

1. Processing of the Test Results.

1.1. Processing of the results made on relative indexes given in relation to the parameters of Test T1. The values of the parameters under the Test T1 are accepted for 100%. The value of relative indexes under the Tests T2 to T7, specified in the Tables below, were determined under the general formula:

Ri=100PTi:PT1,

where Ri—value of a relative index on Test “i”, %; PTi—absolute value of parameter on Test “i”; PT1—absolute value of parameter under the Test T1.

1.2. Mathematical expectation (average arithmetic) MRe and average quadratic deviation σRe were determined under the formulas:

MRe=1/nΣRe; σRe=[1/(n−1)Σ(Re−MRe)²]^{1/2},

where Re—value of a relative index on Test “e” experiment.

1.3. Estimation of confidence intervals: the bottom value Rb and top Rt—was made with confidence probability 0.95 (quantile Up=1,985).

2. Processing of the Test Results.

2.1. The results of definition of relative indexes of parameter PI.Concentration are specified in the Table 1.

TABLE 1

| Experiment | T1 | T2 | T3 | T4 | T5 | T6 | T7 |
|---|---|---|---|---|---|---|---|
| E508 | 100 | 85 | 79 | 63 | 47 | 78 | 56 |
| E509 | 100 | 71 | 56 | 62 | 59 | 69 | 50 |
| E512 | 100 | 51 | 37 | 45 | 36 | 53 | 35 |
| E513 | 100 | 72 | 57 | 54 | 37 | 59 | 48 |
| E516 | 100 | 80 | 75 | 43 | 42 | 53 | 34 |
| E517 | 100 | 83 | 76 | 63 | 48 | 65 | 47 |
| E519 | 100 | 96 | 85 | 87 | 60 | 81 | 47 |
| E520 | 100 | 91 | 72 | 82 | 47 | 87 | 47 |
| E521 | 100 | 92 | 88 | 88 | 47 | 84 | 48 |
| E522 | 100 | 95 | 77 | 81 | 71 | 78 | 46 |
| E523 | 100 | 56 | 52 | 57 | 44 | 58 | 53 |
| E524 | 100 | 65 | 45 | 53 | 32 | 50 | 39 |
| MPI.C | — | 78 | 67 | 65 | 48 | 68 | 46 |
| σPI.C | — | 15.1 | 16.5 | 15.9 | 11.2 | 13.3 | 6.7 |
| σPI.C*Up | — | 30.1 | 32.8 | 31.6 | 22.1 | 26.4 | 13.2 |
| PI.Cb | — | 47.9 | 34.2 | 33.4 | 25.9 | 41.6 | 32.8 |
| PI.Ct | — | 108.1 | 99.8 | 96.6 | 70.1 | 94.4 | 59.2 |

2.2. The results of definition of relative indexes of parameter of the Osmolality are specified in the Table 2.

TABLE 2

| | | | | | | | |
|---|---|---|---|---|---|---|---|
| Experiment | T1 | T2 | T3 | T4 | T5 | T6 | T7 |

| | | | | | | | |
|---|---|---|---|---|---|---|---|
| E508 | 100 | 106 | 101 | 109 | 100 | 110 | 100 |
| E509 | 100 | 105 | 100 | 109 | 100 | 109 | 100 |
| E512 | 100 | 104 | 101 | 109 | 100 | 109 | 100 |
| E513 | 100 | 104 | 100 | 107 | 100 | 108 | 100 |
| E516 | 100 | 105 | 101 | 108 | 100 | 108 | 100 |
| E517 | 100 | 106 | 101 | 110 | 100 | 109 | 101 |
| E519 | 100 | 107 | 99 | 109 | 99 | 110 | 99 |
| E520 | 100 | 106 | 100 | 108 | 99 | 108 | 99 |
| E521 | 100 | 106 | 99 | 109 | 98 | 109 | 98 |
| E522 | 100 | 105 | 99 | 109 | 98 | 109 | 98 |
| E523 | 100 | 105 | 99 | 110 | 99 | 109 | 98 |
| E524 | 100 | 104 | 100 | 107 | 99 | 107 | 98 |
| MOsm | — | 105 | 100 | 109 | 99 | 109 | 99 |
| σOsm | — | 1.0 | 0.9 | 1.0 | 0.8 | 0.9 | 1.1 |
| σOsm*Up | — | 1.9 | 1.7 | 2.0 | 1.5 | 1.7 | 2.1 |
| Osm b | — | 103.1 | 98.3 | 107.0 | 97.5 | 107.3 | 96.9 |
| Osm t | — | 106.9 | 101.7 | 111.0 | 100.5 | 110.7 | 101.1 |

2.3. The results of definition of relative indexes of parameter of Discs are specified in the Table 4.

TABLE 4

| Experiment | T1 | T2 | T3 | T4 | T5 | T6 | T7 |
|---|---|---|---|---|---|---|---|
| E508 | 100 | 15 | single | 7 | single | 10 | single |
| E509 | 100 | 18 | single | 18 | single | 14 | single |
| E512 | 100 | 13 | single | 4 | single | single | single |
| E513 | 100 | 33 | single | 20 | single | 9 | single |
| E516 | 100 | 26 | single | 21 | single | 10 | single |
| E517 | 100 | single | single | single | single | single | single |
| E519 | 100 | 26 | single | 9 | single | 9 | single |
| E520 | 100 | 13 | single | single | single | 6 | single |
| E521 | 100 | 39 | single | 21 | single | 18 | single |
| E522 | 100 | 10 | single | single | single | single | single |
| E523 | 100 | 23 | single | 22 | single | 13 | single |
| E524 | 100 | 27 | single | 21 | single | 14 | single |
| MDiscs | — | 20 | | 12 | | 9 | |
| σDiscs | — | 10.8 | | 9.4 | | 6.0 | |
| σDiscs*Up | — | 21.5 | | 18.7 | | 12.0 | |
| Discs b | — | −1.5 | | −6.7 | | −3.0 | |
| Discs t | — | 41.5 | | 30.7 | | 21.0 | |

2.4. The results of definition of relative indexes of parameter of Dendrites are specified in the Table 5.

TABLE 5

| Experiment | T1 | T2 | T3 | T4 | T5 | T6 | T7 |
|---|---|---|---|---|---|---|---|
| E508 | 100 | 25 | single | 125 | single | 150 | single |
| E509 | 100 | 43 | single | 71 | single | 43 | single |
| E512 | 100 | 75 | single | 150 | single | Single | single |
| E513 | 100 | 75 | single | 150 | single | 125 | single |
| E516 | 100 | 30 | single | 40 | single | 40 | single |
| E517 | 100 | single | single | single | single | single | single |
| E519 | 100 | 75 | single | 150 | single | 125 | single |
| E520 | 100 | 167 | single | single | single | 167 | single |
| E521 | 100 | 57 | single | 29 | single | 57 | single |
| E522 | 100 | 50 | single | single | single | single | single |
| E523 | 100 | 233 | single | 20 | single | 167 | single |
| E524 | 100 | 150 | single | 200 | single | 150 | single |
| MDend | — | 82 | | 78 | | 85 | |
| σDend | — | 67.9 | | 72.7 | | 68.3 | |
| σDend*Up | — | 134.7 | | 144.3 | | 135.6 | |
| Dend b | — | −52.7 | | −66.3 | | −50.6 | |
| Dend t | — | 216.7 | | 222.3 | | 220.6 | |

2.5. The results of definition of relative indexes of parameter of the HSR are specified in the Table 6.

TABLE 6

| Experiment | T1 | T2 | T3 | T4 | T5 | T6 | T7 |
|---|---|---|---|---|---|---|---|
| E508 | 100 | — | 61 | — | 12 | — | 25 |
| E509 | 100 | — | 43 | — | 23 | — | 16 |
| E512 | 100 | — | 113 | — | 23 | — | 58 |
| E513 | 100 | — | 5 | — | 15 | — | 46 |
| E516 | 100 | — | 19 | — | 42 | — | 36 |
| E517 | 100 | — | 22 | — | 13 | — | 32 |
| E519 | 100 | — | 44 | — | 84 | — | 14 |
| E520 | 100 | — | 31 | — | 14 | — | 8 |
| E521 | 100 | — | 23 | — | 2 | — | 14 |
| E522 | 100 | — | 13 | — | 7 | — | 8 |
| E523 | 100 | — | 13 | — | 25 | — | 20 |
| E524 | 100 | — | 3 | — | 7 | — | 27 |
| MHSR | — | | 33 | | 22 | | 25 |
| σHSR | — | | 30.6 | | 22.2 | | 15.5 |
| σHSR*Up | — | | 60.8 | | 44.0 | | 30.7 |
| HSR b | — | | −27.8 | | −22.0 | | −5.7 |
| HSR t | — | | 93.8 | | 66.0 | | 55.7 |

2.6. The results of definition of relative indexes of parameter of the ESC are specified in the Table 7.

TABLE 7

| Experiment | T1 | T2 | T3 | T4 | T5 | T6 | T7 |
|---|---|---|---|---|---|---|---|
| E508 | 100 | — | 0 | — | 0 | — | 29 |
| E509 | 100 | — | 20 | — | 0 | — | 0 |
| E512 | 100 | — | 8 | — | 0 | — | 0 |
| E513 | 100 | — | 14 | — | 14 | — | 0 |
| E516 | 100 | — | 19 | — | 0 | — | 0 |
| E517 | 100 | — | 0 | — | 0 | — | 0 |
| E519 | 100 | — | 38 | — | 31 | — | 23 |
| E520 | 100 | — | 19 | — | 0 | — | 0 |
| E521 | 100 | — | 13 | — | 20 | — | 27 |
| E522 | 100 | — | 14 | — | 14 | — | 0 |
| E523 | 100 | — | 0 | — | 0 | — | 0 |
| E524 | 100 | — | 0 | — | 0 | — | 0 |
| MESC | — | | 12 | | 7 | | 7 |
| σESC | — | | 11.4 | | 13.1 | | 12.0 |
| σESC*Up | — | | 22.7 | | 25.9 | | 23.8 |
| ESC b | — | | −10.7 | | −18.9 | | −16.8 |
| ESC t | — | | 34.7 | | 32.9 | | 30.8 |

2.7. The results of definition of relative indexes of parameter of the optical density Dₒ are specified in the Table 8.

TABLE 8

| Experiment | T1 | T2 | T3 | T4 | T5 | T6 | T7 |
|---|---|---|---|---|---|---|---|
| E508 | 100 | — | 99 | — | 92 | — | 90 |
| E509 | 100 | — | 106 | — | 62 | — | 75 |
| E512 | 100 | — | 94 | — | 66 | — | 52 |
| E513 | 100 | — | 120 | — | 90 | — | 72 |
| E516 | 100 | — | 114 | — | 100 | — | 70 |
| E517 | 100 | — | 114 | — | 103 | — | 79 |
| E519 | 100 | — | 83 | — | 83 | — | 82 |
| E520 | 100 | — | 91 | — | 75 | — | 71 |
| E521 | 100 | — | 108 | — | 137 | — | 129 |
| E522 | 100 | — | 108 | — | 106 | — | 133 |
| E523 | 100 | — | 134 | — | 144 | — | 124 |
| E524 | 100 | — | 99 | — | 75 | — | 72 |
| MDₒ | — | | 106 | | 94 | | 87 |
| σDₒ | — | | 13.9 | | 25.7 | | 26.5 |
| σDₒ*Up | — | | 27.5 | | 51.1 | | 52.5 |
| Dₒ b | — | | 78.5 | | 42.9 | | 34.5 |
| Dₒ t | — | | 133.5 | | 145.1 | | 139.5 |

### EXAMPLE 3

### Summary on Stages E(544-548)

In this example, the possibility of saving discoid platelets and the platelet functionality in gelling solution containing starch—1.4%, sucrose—1.4%; and PVP—1%, 2%, 3% at t+2C by cooling down to t+2C at the rate 1 degree C./sec. under atmospheric pressure, was investigated.

Solutions, Conditions, Test Results

The PC for experiments was prepared as follows:

The whole blood collected from one donor was centrifuged at acceleration 1740 g for 4 minutes at t+22C. The PRP was extracted in a satellite bag and was centrifuged at acceleration 4323 g for 6 minutes at temperature +22C. The supernatant layer of the plasma was excreted and the volume of suspended plasma with the platelet plaque made 60 ml. The bags with plasma containing platelet plaques were placed on the rocker at t+22C, where they were rested for 20 to 22 hours. These bags were assayed and the platelet concentration in the PC was identified. Then the assay taken from the PC (Test 1) was tested. The bags were added the verapamil solution, so that the final concentration made 5 mg/L. The bags were placed on the rocker at t+22C, where they were rested for 30 minutes. Then the PC was added the solution of MgCl2 so that its final concentration made 0.95 g/L (10 mM). Then each bag with the PC collected from one donor was split into 3 sets of bags, each containing 20 ml.

The bags with Numbers 1 were added 2.25 volumes of an additive solution containing starch—2%, sucrose—2%; and PVP(12600±1400 Da)—1.45%, in a solution “Transvect.” In view of dilution the concentrations, the concentration of HES and sucrose were 1.4% and that of PVP—2%. The osmolality of this PC solution made 329 to 333 mOsm/L.

The bags with Numbers 2 were added 2.25 volumes of an additive solution containing starch—2%, sucrose—2%; and PVP(12600±1400 Da)—2%, in a solution “Transvect.” In view of dilution the concentrations the concentration of HES and sucrose were 1.4% and that of PVP—2%. The osmolality of this PC solution made 340 to 343 mOsm/L.

The bags with Numbers 3 were added 2.25 volumes of an additive solution containing starch—2%, sucrose—2%; and PVP(12600±1400 Da)—4.35%, in a solution “Transvect.” In view of dilution the concentrations the concentration of HES and sucrose were 1.4% and that of PVP—3%. The osmolality of this PC solution made 357 to 361 mOsm/L.

The final concentration of inhibitors made: verapamil—1.55 mg/L; MgCl2—0.29 g/L (3.1 mM).

The PC with the additive solutions from the bags was distilled in satellite bags through the silicon drainage tube being in a bath with a cooled saline solution. The satellite bags were in an ice bath at temperature +1C, and the silicon drainage tube was in a saline solution at temperature −8.5C. The temperature of the PC with an additive solution was controlled at the output from the silicon drainage tube with the help of the thermocouple. This temperature made +0.5C to +4C. The rates of cooling of solutions from +18C down to +6c made 0.99 to 1.21 degree C./sec. (average rate is 1.04 degree C./sec.), and average cooling-off period of solutions in the specified interval of temperatures—11.5 seconds. Then these bags were transferred to the incubator at temperature +2C.

After storage for 24 hours the bags with Numbers 1, 2 and 3 were warmed up to t+22C in the water bath and assayed for testing: Test 2 for the bags with Numbers 1, Test 4 for the bags with Numbers 2, and Test 6 for the bags with Numbers 3. The tubes with the assays were placed on a bay at a room temperature (t+25.5C) were they were rested for 3-4 hours before the percent of the platelet shape was determined. During this time, the assay were test with solutions: platelet concentration and osmolality and pH of solutions, and also testing of the washed out platelet-HSR and ESC, except for the already specified parameters. The assays taken from bags were centrifuged at acceleration 1460 g for 10 minutes.

The supernatant layer of plasma was excreted and the volume was brought in to the stock volume with the donor plasma. The PRP assays were placed on the rocker at temperature +22C, where they were rested for 1 hour. Then these assays were tested: Test 3 for bags with Numbers 1, Test 5 for bags with Numbers 2, Test 7 for bags with Numbers 3.

The microscopy of the assays taken from bags with Numbers 1 demonstrated that at a room temperature the gel survived in the solutions. Therefore, it was difficult to analyze disks in such solution with high viscosity.

Some microfragments, single platelet aggregates, dendrites and altered disks were found in these assays, though there were no platelet aggregates. The discoid platelets have both perfect and altered shapes. The relation between perfect and altered discs were about 1:4. The altered discs were oblong and convex, many of them had one pseudopodium each. Besides, in some assays taken from these bags, some conglutinated platelets were found. After centrifugation and washing out of the platelet, microfragments and small platelet aggregates were found almost in each assay.

The ANALYSIS Supplement immediately below shows a statistical analysis of data obtained for the platelet concentration, osmolality and pH of solutions, percents of disks, HSR and ESC and optical densities Dₒ.

The average values of the platelet parameters expressed in percentage in relation to these parameters in the stock PRP are submitted in the Table below. And the stock platelet concentrations in all bags were so that after getting in 2.25 volumes of an additive solution in the PC the platelet count has depressed down to 0.31. The Summary Table shows the average value of the platelet count, percents of disks, HSR and ESC in all bags after storage at temperature +2C. Confidence intervals appropriate to confidence probability are specified there 0.95. Thus, a significance level of the received parameters p≦0.05, i.e. with the probability 0.05 the measured parameters can fall outside the range of confidence intervals.

Summary Table: E(544-548); p ≦ 0.05.

| | Concentration, | Discs, | HSR, | ESC, |
|---|---|---|---|---|
| | % | % | % | % |
| Bag 1: | 74 ± 24.0 | 39 ± 33.1 | 90 ± 39.4 | 18 ± 33.4 |
| Starch-1.4%, | | | | |
| Sucrose-1.4%, | | | | |
| PVP-1% | | | | |
| Bag 2: | 90 ± 11.5 | 39 ± 35.3 | 83 ± 73.6 | 21 ± 24.6 |
| Starch-1.4%, | | | | |
| Sucrose-1.4%, | | | | |
| PVP-2.%. | | | | |
| Bag 3: | 85 ± 19.3 | 44 ± 23.7 | 84 ± 79.0 | 27 ± 8.4 |
| starch-1.4%, | | | | |
| sucrose-1.4%, | | | | |
| PVP-3.% | | | | |

As is visible from these data in a solution containing 1.4% of HES, 1.4% of sucrose and 3% PVP (bag 3), the average percents of disks and ESC were the highest, the average concentration of the platelets and HSR does not differ much from their maximal values.

The dropping of the platelet concentration in a bag 1 may have been caused by small concentration of PVP that did not save the integrity of the platelet membranes after cooling at the rate 1 deg. C./sec. And the platelet concentration in a bag 1 was caused, apparently, by elevated concentration of PVP, at which a viscid gel formed in the solution PC, that deformed a liquid crystal membranes of the platelets after cooling down to +2C.

### Analysis

Test Results of the Stages E(544-548) Experiments, made Under Invariable Conditions

1. Processing of the Test Results.

1.1. Processing of the results made on relative indexes given in relation to the parameters of Test T1. The values of the parameters under the Test T1 are accepted for 100%. The value of relative indexes under the Tests T2 . . . T7, specified in the Tables below, were determined under the general formula:

Ri=100PTi:PT1,

where Ri—value of a relative index on Test “i”, %; PTi—absolute value of parameter on Test “i”; PT1—absolute value of parameter under the Test T1.

1.2. Mathematical expectation (average arithmetic) MRe and average quadratic deviation σRe were determined under the formulas:

MRe=1/nΣRe; σRe=[1/(n−1)Σ(Re−MRe)²]^{1/2},

where Re—value of a relative index on Test “e” experiment.

1.3. Estimation of confidence intervals: the bottom value Rb and top Rt—was made with confidence probability 0.95 (quantile Up=1,985).

2. Processing of the Test Results.

2.1. The results of definition of relative indexes of parameter PI.Concentration are specified in the Table 1.

TABLE 1

| Experiment | T1 | T2 | T3 | T4 | T5 | T6 | T7 |
|---|---|---|---|---|---|---|---|
| E544 | 100 | 65 | 56 | 90 | 50 | 73 | 64 |
| E545 | 100 | 77 | 54 | 90 | 60 | 79 | 55 |
| E546 | 100 | 61 | 41 | 83 | 52 | 83 | 53 |
| E547 | 100 | 92 | 58 | 88 | 54 | 90 | 59 |
| E548 | 100 | 75 | 66 | 99 | 63 | 98 | 69 |
| MPI.C | — | 74 | 55 | 90 | 56 | 85 | 60 |
| σPI.C | — | 12.1 | 9.1 | 5.8 | 5.5 | 9.7 | 6.6 |
| σPI.C*Up | — | 24.0 | 18.0 | 11.5 | 10.9 | 19.3 | 13.0 |
| PI.Cb | — | 50.0 | 37.0 | 78.5 | 45.1 | 65.7 | 47.0 |
| PI.Ct | — | 98.0 | 73.0 | 101.5 | 66.9 | 104.3 | 73.0 |

2.2. The results of definition of relative indexes of parameter of the Osmolality are specified in the Table 2.

TABLE 2

| Experiment | T1 | T2 | T3 | T4 | T5 | T6 | T7 |
|---|---|---|---|---|---|---|---|
| E544 | 100 | 107 | 100 | 111 | 101 | 116 | 102 |
| E545 | 100 | 105 | 100 | 108 | 100 | 114 | 100 |
| E546 | 100 | 106 | 100 | 110 | 100 | 116 | 101 |
| E547 | 100 | 107 | 100 | 111 | 100 | 116 | 101 |
| E548 | 100 | 108 | 100 | 111 | 101 | 118 | 102 |
| MOsm | — | 107 | 100 | 110 | 100 | 116 | 101 |
| σOsm | — | 1.1 | 0 | 1.3 | 0.5 | 1.4 | 0.6 |
| σOsm*Up | — | 2.3 | 0 | 2.6 | 1.1 | 2.8 | 1.7 |
| Osm b | — | 104.7 | 100 | 107.4 | 98.9 | 113.2 | 99.3 |
| Osm t | — | 109.3 | 100 | 112.6 | 101.1 | 118.8 | 102.7 |

2.3. The results of definition of relative indexes of parameter of the pH are specified in the Table 3.

TABLE 3

| Experiment | T1 | T2 | T3 | T4 | T5 | T6 | T7 |
|---|---|---|---|---|---|---|---|
| E544 | 100 | 104 | 105 | 104 | 104 | 103 | 104 |
| E545 | 100 | 103 | 104 | 103 | 103 | 102 | 103 |
| E546 | 100 | 105 | 106 | 105 | 106 | 105 | 106 |
| E547 | 100 | 112 | 114 | 112 | 114 | 112 | 114 |
| E548 | 100 | 101 | 103 | 101 | 102 | 101 | 101 |
| MpH | — | 105 | 106 | 105 | 106 | 105 | 106 |
| σpH | — | 4.8 | 4.4 | 4.2 | 4.8 | 4.4 | 5.0 |
| σpH*Up | — | 8.3 | 8.7 | 8.3 | 9.6 | 8.7 | 10.0 |
| pHb | — | 96.7 | 97.3 | 96.7 | 96.4 | 96.3 | 96.0 |
| pHt | — | 113.3 | 114.7 | 113.3 | 115.6 | 113.7 | 116.0 |

2.4. The results of definition of relative indexes of parameter of Discs are specified in the Table 4.

TABLE 4

| Experiment | T1 | T2 | T3 | T4 | T5 | T6 | T7 |
|---|---|---|---|---|---|---|---|
| E544 | 100 | 45 | single | 35 | single | 40 | single |
| E545 | 100 | 18 | single | 22 | single | 31 | single |
| E546 | 100 | 32 | single | 33 | single | 60 | single |
| E547 | 100 | 63 | single | 67 | single | 52 | single |
| E548 | 100 | 36 | single | 38 | single | 36 | single |
| MDiscs | — | 39 | | 39 | | 44 | |
| σDiscs | — | 16.7 | | 16.8 | | 11.9 | |
| σDiscs*Up | — | 33.1 | | 33.3 | | 23.7 | |
| Discs b | — | 5.9 | | 5.7 | | 20.3 | |
| Discs t | — | 72.1 | | 72.3 | | 67.7 | |

2.5. The results of definition of relative indexes of parameter of Dendrites are specified in the Table 5.

TABLE 5

| Experiment | T1 | T2 | T3 | T4 | T5 | T6 | T7 |
|---|---|---|---|---|---|---|---|
| E544 | 100 | 50 | single | 80 | single | 40 | single |
| E545 | 100 | 100 | single | 120 | single | 60 | single |
| E546 | 100 | 300 | single | 250 | single | 150 | single |
| E547 | 100 | 133 | single | 167 | single | 267 | single |
| E548 | 100 | 400 | single | 400 | single | 300 | single |
| MDend | — | 197 | | 203 | | 163 | |
| σDend | — | 147.4 | | 126.8 | | 117.8 | |
| σDend*Up | — | 292.6 | | 251.7 | | 233.8 | |
| Dend b | — | 95.6 | | −48.7 | | −70.8 | |
| Dend t | — | 489.6 | | 454.7 | | 396.8 | |

2.6. The results of definition of relative indexes of parameter of the HSR are specified in the Table 6.

TABLE 6

| Experiment | T1 | T2 | T3 | T4 | T5 | T6 | T7 |
|---|---|---|---|---|---|---|---|
| E544 | 100 | — | 106 | — | 127 | — | 125 |
| E545 | 100 | — | 106 | — | 102 | — | 47 |
| E546 | 100 | — | 99 | — | 49 | — | 81 |
| E547 | 100 | — | 63 | — | 97 | — | 44 |
| E548 | 100 | — | 74 | — | 40 | — | 125 |
| MHSR | — | | 90 | | 83 | | 84 |
| σHSR | — | | 19.9 | | 37.1 | | 39.8 |
| σHSR*Up | — | | 39.4 | | 73.6 | | 79.0 |
| HSR b | — | | 50.6 | | 9.4 | | 5.0 |
| HSR t | — | | 129.4 | | 156.6 | | 163.0 |

2.7. The results of definition of relative indexes of parameter of the ESC are specified in the Table 7.

TABLE 7

| Experiment | T1 | T2 | T3 | T4 | T5 | T6 | T7 |
|---|---|---|---|---|---|---|---|
| E544 | 100 | — | 0 | — | 0 | — | 30 |
| E545 | 100 | — | 30 | — | 30 | — | 30 |
| E546 | 100 | — | 0 | — | 20 | — | 27 |
| E547 | 100 | — | 35 | — | 24 | — | 29 |
| E548 | 100 | — | 25 | — | 30 | — | 20 |
| MESC | — | | 18 | | 21 | | 27 |
| σESC | — | | 16.8 | | 12.4 | | 4.2 |
| σESC*Up | — | | 33.4 | | 24.6 | | 8.4 |
| ESC b | — | | −15.4 | | −3.6 | | 18.6 |
| ESC t | — | | 51.4 | | 45.6 | | 35.4 |

2.8. The results of definition of relative indexes of parameter of the optical density Dₒ are specified in the Table 8.

TABLE 8

| Experiment | T1 | T2 | T3 | T4 | T5 | T6 | T7 |
|---|---|---|---|---|---|---|---|
| E544 | 100 | — | 127 | — | 132 | — | 116 |
| E545 | 100 | — | 103 | — | 89 | — | 90 |
| E546 | 100 | — | 95 | — | 102 | — | 140 |
| E547 | 100 | — | 133 | — | 132 | — | 116 |
| E548 | 100 | — | 107 | — | 108 | — | 108 |
| MDₒ | — | | 113 | | 113 | | 114 |
| σDₒ | — | | 16.2 | | 19.0 | | 18.0 |
| σDₒ*Up | — | | 32.3 | | 37.7 | | 35.7 |
| Dₒ b | — | | 80.7 | | 75.3 | | 78.3 |
| Dₒ t | — | | 145.3 | | 150.7 | | 149.7 |

While the invention has been described with reference to the aforementioned specification, the descriptions and illustrations of the preferred embodiments herein are not meant to be construed in a limiting sense. It shall be understood that all aspects of the invention are not limited to the specific depictions, configurations or relative proportions set forth herein which depend upon a variety of conditions and variables. It is not intended to be exhaustive or to limit the invention to the precise forms disclosed. Many modifications and variations will be apparent. Various modifications in form and detail of the embodiments of the invention, as well as other variations of the invention, will be apparent to a person skilled in the art upon reference to the present disclosure. It is therefore contemplated that the appended claims shall cover any such modifications, variations or equivalents of the described embodiments as falling within the true spirit and scope of the invention.

What is claimed is:

1. A method for preserving platelets comprising the following steps of:
preparing a platelet suspension having blood platelets, a carbohydrate and at least one biocompatible polymer to assist in stabilizing platelet membranes;
cooling the platelet suspension to a temperature of less than approximately 10 degrees C. at a rate of cooling greater than 1 degree C./min; and
storing the platelet suspension at a storage temperature ranging from approximately −1 to 6 degrees C.

2. The method as recited in claim 1 wherein the platelet suspension is cooled to a temperature 6 degrees C.

3. The method as recited in claim 1 wherein the rate of cooling is at least 12 degrees C./min.

4. The method as recited in claim 1 wherein the carbohydrate is selected from the group consisting of glucose and sucrose.

5. The method as recited in claim 1 wherein the platelet suspension further comprises at least one membrane stabilizing agent to assist in stabilizing platelet membranes.

6. The method as recited in claim 5 wherein the suspension includes a plurality of membrane stabilizing agents including sucrose and magnesium.

7. A method for reducing membrane phase transition in blood platelets comprising the following steps of:
selecting a medium containing platelets, a sugar, and a biocompatible gelling agent for stabilizing platelet membranes, wherein the medium has an initial temperature above about 18 degrees C.;
reducing the initial temperature of the medium to a transition temperature below approximately 14 degrees C. at a first cooling rate greater than about 1 degree C./min;
further reducing the transition temperature of the medium to a storage temperature below approximately 6 degrees C. at a second cooling rate greater than about 1 degree C./min; and
storing the medium at a storage temperature of less than about 6 degrees C.

8. The method as recited in claim 7 wherein the first cooling rate and the second cooling rate are the same.

9. The method as recited in claim 7 wherein the first and second cooling rates are 120 degrees C./min.

10. The method as recited in claim 7 wherein the medium further comprises verapamil and magnesium.

11. The method as recited in claim 7 wherein the initial concentration of verapamil has a range of up to 3 mg/L and the magnesium has a range from 1 to 10 mM.

12. The method as recited in claim 7 wherein the osmolarity of the medium ranges from about 300 mOsm to 380 mOsm.

13. A method for maintaining the biological activity of blood platelets comprising the following steps of:
forming a platelet suspension having platelets, sucrose, verapamil, magnesium chloride and a polymer selected from the group consisting of hydroxyethyl starch, polyvinylpyrrolidone and dextran,
cooling the platelet suspension at a cooling rate ranging from approximately 1 to 12 degrees C./min to a temperature below 10 degrees C.; and
storing the cooled platelet suspension at a temperature no higher than 6 degrees C.

14. The method as recited in claim 13 wherein the platelet suspension is cooled within one hour after the platelet suspension is formed.

15. The method are recited in claim 13 wherein the cooled platelet suspension is stored for at least ten days.

16. The method as recited in claim 13 wherein the final concentrations in the platelet suspension for the verapamil is 5 mg/L, the magnesium chloride is 10 mM, the sucrose is 1.4%, and the polymer is 1.4%.

17. The method as recited in claim 13 further comprising the steps of:
warming the cooled platelet suspension to about 20 degrees C. at a warming rate of about 2 degrees C.; and
infusing the platelet suspension into the bloodstream of a patient.
